(19) United States
(12) Patent Application Publication
(10) Pub. No.: US 2005/0169870 A1
(43) Pub. Date: Aug. 4, 2005

(54) Methods of modulating CD200

(75) Inventors: Robert L. Truitt, Germantown, WI (US); Michael Rosenblum, Milwaukee, WI (US); Edit Olasz, Wauwatosa, WI (US)

Correspondence Address: DNAX RESEARCH, INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 11/047,456

(22) Filed: Jan. 31, 2005

Related U.S. Application Data
(60) Provisional application No. 60/541,082, filed on Feb. 2, 2004.

(51) Int. Cl. A61K 39/395; A61K 38/17; A61K 7/06; A61K 31/59; A61K 7/11

(52) U.S. Cl. 424/70.14; 424/144.1; 514/2

(57) ABSTRACT

Provided are methods for modulating activity of the immune system using agonists or antagonists of CD200 pr CD200R. Also provided are methods of treatment and diagnosis of immune disorders.

This filing is a U.S. Patent Application which claims benefit of U.S. Provisional Patent Application No. 60/541,082, filed Feb. 2, 2004, which is incorporated herein by reference.

### FIELD OF THE INVENTION

The present invention relates to methods and compositions for modulating mammalian physiology, including immune system function. In particular, it provides methods for modulating activities dependent on CD200 and CD200R. Diagnostic and therapeutic uses are disclosed.

### BACKGROUND OF THE INVENTION

The immune system functions to protect individuals from infective agents, e.g., bacteria, multi-cellular organisms, and viruses, as well as from cancers. This system includes several types of lymphoid and myeloid cells, e.g., monocytes, macrophages, dendritic cells (DCs), eosinophils, T cells, B cells, and neutrophils. These lymphoid and myeloid cells often produce soluble signaling proteins known as cytokines. The immune response includes inflammation, i.e., the accumulation of immune cells systemically or in a particular location of the body. In response to an infective agent or foreign substance, or in an autoimmune response, immune cells secrete cytokines which, in turn, modulate immune cell proliferation, development, differentiation, or migration. Membrane-bound proteins also mediate signaling in immune response. For example, T cell receptor, CD4, B cell receptor, CD20, and FcgammaRIII are membrane-bound proteins that transmit activating signals, while CD200 (also known as OX2) and its corresponding receptor CD200R (a.k.a. OX2R), as well as CTLA-4, CD94, SIRPs, and FcgammaRIIb, transmit inhibitory signals to the cell (see, e.g., Abbas, et al. (eds.) (2000) Cellular and Molecular Immunology, W.B. Saunders Co., Philadelphia, Pa.; Oppenheim and Feldmann (eds.) (2001) Cytokine Reference, Academic Press, San Diego, Calif.; von Andrian and Mackay (2000) New Engl. J. Med. 343:1020-1034; Davidson and Diamond (2001) New Engl. J. Med. 345:340-350; Nathan and Muller (2001) Nature Immunology 2:17-19).

The present invention provides a method to treat alopecia, e.g., non-scarring alopecia and scarring alopecia. Scarring alopecia tends to involve permanent loss of hair follicles, while non-scarring alopecia may involve reversible follicular loss. Non-scarring alopecias include androgenetic alopecia (AGA), alopecia areata (AA), traction alopecia (TA), and frontal fibrosing alopecia. Alopecia areata (AA), a non-scarring, inflammatory hair loss disorder, is a common form of hair loss accounting for about 2% of dermatology patients in the United States, and results in baldness in adults and children. Androgenetic alopecia is the most common type of hair loss in men, while changes in androgen metabolism can also contribute to female pattern hair loss. Although these disorders are non-scarring, permanent follicular loss can occur in later stages of AGA, AA, and TA. In other words, the disorders AGA, AA, and TA can show a biphasic pattern.

Scarring alopecia, an alopecia where hair destruction occurs early in the course of the disease, takes several forms, e.g., pseudopelade of Brocq (PB), chronic, cutaneous lupus erythematosus (CCLE), lichen planopilaris (LPP), dissecting cellulites, acne keloidalis, central, centrifugal scarring alopecia (CCSA), and fibrosing alopecia. PB, which involves numerous patches in the scalp that coalesce into larger, irregular plaques, can occur as a stage of LPP or discoid lupus erythematosus (DLE). CCSA, which involves hair loss centered at the top of the scalp, encompasses follicular degeneration syndrome, pseudopelade, folliculitis decalvans, and tufted folliculitis. Lichen planopilaris, also known as lichen planus pilaris (LPP), involves several scattered foci of hair loss, and encompasses Graham-Little syndrome and frontal fibrosing alopecia. CCLE involves a scaly plaque and can be a manifestation of systemic lupus erythematosus (SLE).

Scarring alopecia includes disorders where hair follicles are specifically destroyed by inflammatory processes, but also disorders where hair follicles are destroyed as a side-effect of nearby inflammation, where these latter disorders include, e.g., cutaneous sarcoid, morphea, necrobiosis lipoidica, lupus vulgaris, and the like (see, e.g., McElwee and Hoffinann (2002) Clin. Exp. Dermatol. 27:410-417; Sperling (2001) J. Cutaneous Pathol. 28:333-342; Sperling, et al. (2000) Arch. Dermatol. 136:235-242; Zinkemagel, et al. (2000) Arch. Dermatol. 136:205-211; Amato, et al. (2002) Int. J. Dermatol. 41:8-15; Hoffinan (2002) Clin. Exp. Dermatol. 27:373-382; Birch, et al. (2002) Clin. Exp. Dermatol. 27:383-388).

A number of observations have demonstrated an immune component in alopecia, e.g., in AA, AGA, PB, LPP, and CCSA. Alopecia is characterized by infiltration or activation of immune cells, e.g., macrophages, T cells, mast cells, neutrophils, Langerhans cells, or eosinophils. Studies of alopecia areata have demonstrated that most of these infiltrating immune cells cells are perifollicular and in the hair sheath. Changes in the activation state of hair follicle epithelial cells, such as, keratinocytes, also promote immune response, e.g., by increased expression of cell adhesion molecules and increased expression of follicular autoantigen.

In addition to immune cell infiltration, cytokine expression contributes to the pathology of alopecia, as shown by studies of, e.g., IL-1beta, interferon-gamma (IFNgamma), IL-2, IL-6, and IL-10. Moreover, increased expression of the pro-inflammatory neurotransmitter, substance P, has been found in alopecia areata (see, e.g., Elston, et al. (2000) J. Am. Acad. Dermatol. 37:101-106; El Darouti, et al. (2000) J. Am. Acad. Dermatol. 42:305-307; Bodemer, et al. (2000) J. Invest. Dermatol. 114:112-116; Gilhar, et al. (1998) J. Clin. Invest. 101:62-67; McElwee, et al. (1996) Br. J. Dermatol. 135:211-217; Toyoda, et al. (2001) Br. J. Dermatol. 144:46-54; Sullivan and Kossard (1998) Australas J. Dermatol. 39:207-218; Hoffmann, et al. (1994) J. Invest. Dermatol. 103:530-533; Price (2003) J. Invest. Dermatol Symp. Proc. 8:207-211; Sperling, et al. (2001) J. Cutaneous Pathol. 28:333-342; Millikan (2001) Int. J. Dermatol. 40:475-476; Mahe, et al. (2000) Int. J. Dermatol. 39:576-584; Young, et al. (1991) J. Am. Osteopath. Assoc. 91:765-771; Amato, et al. (2002) Int. J. Dermatol. 41:8-15).

Intervention studies have also demonstrated an immune component of alopecia areata. Depleting CD8+ T cells results in an amelioration of alopecia, while injecting CD8+ T cells results in the acquisition of alopecia. In another approach, the blocking of a specific activating receptor of immune cells (CD44) was shown to be an effective treatment of alopecia (Hoffinann (1999) J. Investig. Dermatol. Symp. Proc. 4:235-238; Kalish and Gilhar (2003) J. Investig. Dermatol. Symp. Proc. 8:164-167; Tsuboi, et al. (1999) J. Dermatol. 26:797-802; Zoller, et al. (2002) J. Invest. Dermatol. 118:983-992).

Alopecia is a poorly understood disorder and the available treatments are not fully effective. The present invention fulfills this need by providing methods of treatment and diagnosis, e.g., using agonists and antagonists of CD200.

### SUMMARY OF THE INVENTION

The invention is based, in part, upon the discovery that CD200 can inhibit alopecia.

The present invention provides a method of treating a condition or disorder associated with a hair follicle comprising administering to a subject an effective amount of an agonist of CD200 or CD200R. Also provided is the above method, wherein the agonist is from the antigen binding site of an antibody that specifically binds to CD200 or CD200R. In another aspect, the invention provides the above method wherein the agonist comprises: a polyclonal antibody; a monoclonal antibody; a humanized antibody, or a fragment thereof; an Fab, F(ab′)₂, or Fv fragment; a bispecific antibody; a peptide mimetic of an antibody; or a small molecule. Also provided is the above method wherein the bispecific antibody specifically binds CD200R and an activating receptor; and the above method wherein the agonist comprises: a soluble polypeptide derived from an extracellular region of CD200, wherein the soluble polypeptide specifically binds to CD200R; or a soluble polypeptide derived from an extracellular region of CD200R, wherein the soluble polypeptide specifically binds to CD200.

Another embodiment of the present invention provides a method of treating a condition or disorder of a hair follicle comprising administering to a subject an effective amount of an agonist of CD200 or CD200R, wherein the agonist or antagonist comprises a nucleic acid; wherein the nucleic acid encodes: CD200 or CD200R; or a soluble polypeptide derived from an extracellular region of CD200 or an extracellular region of CD200R.

Yet another aspect of the present invention provides a method of treating a condition or disorder of a hair follicle comprising administering to a subject an effective amount of an agonist of CD200 or CD200R; wherein the condition or disorder comprises alopecia and the agonist ameliorates the alopecia or increases hair growth; wherein the alopecia comprises scarring alopecia or non-scarring alopecia; the above method wherein the alopecia comprises: androgenetic alopecia (AGA); alopecia areata (AA); pseudopelade of Brocq (PB); lichen planopilaris (LPP); or fibrosing alopecia (FA); as well as the above method wherein the condition or disorder comprises: hair loss or baldness; fibrosis in a dermal layer of the hair follicle; intrafollicular edema; apoptosis of a cell of the hair follicle; infiltration of the hair follicle by an immune cell; hair follicle depigmentation; or excess hair.

Moreover, the present invention provides a method of treating a condition or disorder of a hair follicle comprising administering to a subject an effective amount of an agonist of CD200 or CD200R, wherein the agonist results in increased expression of insulin-like growth factor-1 or interferon-gamma; the above method wherein the CD200 is expressed by: an outer root sheath; a keratinocyte; a Langerhans cell; a keratin-14 expressing cell; or a hair follicle stem cell or transit amplifying cell; as well as the above method wherein the disorder is alopecia and the agonist ameliorates the alopecia or increases hair growth.

The present invention provides for treatment of a disorder or condition associated with excess hair growth comprising administering an antagonist of CD200 or CD200R to reduce or inhibit hair growth.

In another embodiment, the present invention provides a method of diagnosing a condition or disorder of a hair follicle comprising contacting a binding composition to a biological sample, wherein the binding composition specifically binds to: CD200 or CD200R, and measuring or determining the specific binding of the binding composition to the biological sample, as well as the above method wherein the biological sample is derived from a hair follicle of: a tissue afflicted with a condition or disorder of the hair follicle; or a control subject or non-afflicted tissue. Also provided is a kit comprising a compartment and: the agonist of CD200 or CD200R or a nucleic acid that specifically hybridizes to a polynucleotide encoding CD200 or CD200R.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual publication, patent application, or patent, was specifically and individually indicated to be incorporated by reference.

I. Definitions.

“Activity” of a molecule may describe or refer to binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. Treatment encompasses methods using a purified immune cell, e.g., in a mixed cell reactions or for administration to a research, animal, or human subject. The invention contemplates treatment with a cell, a purified cell, a stimulated cell, a cell population enriched in a particular cell, and a purified cell. Treatment further encompasses situations where an administered reagent or administered cell is modified by metabolism, degradation, or by conditions of storage.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, including selenomethionine, as well as those amino acids that are modified after incorporation into a polypeptide, e.g., hydroxyproline, O-phosphoserine, O-phosphotyrosine, gamma-carboxyglutamate, and cystine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetic refers to a chemical compound that has a structure that is different from the general chemical structure of an amino acid, but that functions in a manner similar to a naturally occurring amino acid. Amino acids may be referred to herein by either their commonly known three letter symbols or by their one-letter symbols.

“Binding composition” refers to a molecule, small molecule, macromolecule, antibody, a fragment or analogue thereof, or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution.

“Bispecific antibody” generally refers to a covalent complex, but may refer to a stable non-covalent complex of binding fragments from two different antibodies, humanized binding fragments from two different antibodies, or peptide mimetics derived from binding fragments from two different antibodies. Each binding fragment recognizes a different target or epitope, e.g., a different receptor, e.g., an inhibiting receptor and an activating receptor. Bispecific antibodies normally exhibit specific binding to two different antigens.

“Conservatively modified variants” applies to both amino acid and nucleic acid sequences. With respect to particular nucleic acid sequences, conservatively modified variant refers to those nucleic acids that encode identical or essentially identical amino acid sequences. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132).
(1) Hydrophobic: Norleucine, Ile, Val, Leu, Phe, Cys, Met;
(2) Neutral hydrophilic: Cys, Ser, Thr;
(3) Acidic: Asp, Glu;
(4) Basic: Asn, Gln, His, Lys, Arg;
(5) Residues that influence chain orientation: Gly, Pro;
(6) Aromatic: Trp, Tyr, Phe; and
(7) Small amino acids: Gly, Ala, Ser.

Methods relating to polypeptide molecules having substantially the same amino acid sequence as CD200 or CD200R but possessing minor amino acid substitutions, truncations, or deletions, that do not substantially affect the functional aspects are within the definition of the contemplated invention. Variants containing one or more peptide bond cleavages, where daughter polypeptides remain in association with each other, are within the definition of the contemplated invention.

Endpoints in activation or inhibition can be monitored as follows. Activation, inhibition, and response to treatment, e.g., of a cell, hair follicle, keratinocyte, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

“Exogenous” refers to substances that are produced outside an organism, cell, or human body, depending on the context. “Endogenous” refers to substances that are produced within a cell, organism, or human body, depending on the context.

A “marker” relates to the phenotype of a cell, tissue, organ, animal, or human subject. Markers are used to detect cells, e.g., during cell purification, quantitation, migration, activation, maturation, or development, and may be used for both in vitro and in vivo studies. An activation marker is a marker that is associated with cell activation.

“Monofunctional reagent” refers, e.g., to an antibody, binding composition derived from the binding site of an antibody, an antibody mimetic, a soluble receptor, engineered, recombinant, or chemically modified derivatives thereof, that specifically binds to a single type of target. For example, a monofunctional reagent may contain one or more functioning binding sites for a CD200 receptor. “Monofunctional reagent” also refers to a polypeptide, antibody, or other reagent that contains one or more functioning binding sites for, e.g., CD200 receptor and one or more non-functioning binding sites for another type of receptor. For example, a monofunctional reagent may comprise an antibody binding site for CD200 receptor plus an Fc fragment that has been engineered so that the Fc fragment does not specifically bind to Fc receptor.

“Nucleic acid” refers to deoxyribonucleotides or ribonucleotides and polymers thereof in either single stranded or double-stranded form. The term nucleic acid may be used interchangeably with gene, cDNA, mRNA, oligonucleotide, and polynucleotide. A particular nucleic acid sequence also implicitly encompasses “allelic variants” and “splice variants.”

“Condition” of a hair follicle encompasses disorders but also states of the hair follicle that are not necessarily classified as disorders, e.g., cosmetic conditions or states of normal physiology. Disorders of a hair follicle encompasses disorders of a cell, where the cell is in the same genetic lineage of a hair follicle cell, e.g., a precursor cell of a hair follicle keratinocyte where the precursor is committed to becoming a keratinocyte.

“Sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

Small molecules are provided for the treatment of physiology and disorders of the hair follicle. “Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecule toxins are described (see, e.g., U.S. Pat. No. 6,326,482 issued to Stewart, et al).

“Specifically” or “selectively” binds, when referring to a ligand/receptor, antibody/antigen, or other binding pair, indicates a binding reaction which is determinative of the presence of the protein in a heterogeneous population of proteins and other biologics. us, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity or binding constant that is at least two fold greater, preferably at least ten times greater, more preferably at least 20-times greater, and most preferably at least 100-times greater than the affinity with any other antibody, or binding composition derived thereof. In a preferred embodiment the antibody will have an affinity that is greater than about 10⁹ liters/mol, as determined, e.g., by Scatchard analysis (Munsen, et al. (1980) Analyt. Biochem. 107:220-239).

“Tolerance” involves failure of the immune system to mount a response to an antigen. “Immune privilege” is a form of tolerance, where tolerance results, e.g., because the antigen resides in a site that is not accessable to immune cells (Kamradt and Mitchison (2001) New Engl. J. Med. 344:655-664; Waldmann and Cobbold (1998) Annu. Rev. Immunol. 16:619-644; Ohashi and DeFranco (2002) Curr. Opinion Immunol. 14:744-759; Liu (1997) J. Exp. Med. 186:625-629; Wood and Sakaguchi (2003) Nature Revs. Immunology 3:199-210; Christoph, et al. (2000) Br. J. Dermatol. 142:862-873; Paus, et al. (2003) J. Investig. Dermatol. Symp. Proc. 8:188-194; Taylor (2003) Ocul. Immunol. Inflamm. 11:231-241; Ferfuson, et al. (2002) Int. Rev. Immunol. 21:153-172; Steinman, et al. (2003) Annu. Rev. Immunol. 21:685-711; Streilein and Stein-Streilein (2000) J. Leukocyte Biol. 67:479-487).

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of a CD200 agonist, such as a soluble version of CD200 or an agonistic antibody to CD200R, or an antagonist of CD200, to a human or animal subject, or to a cell, tissue, physiological compartment, or physiological fluid. “Treatment of a cell, tissue, organ, or subject” encompasses situations where it has not been demonstrated that the agonist or antagonist of CD200 has contacted CD200R, or where it has not been demonstrated that the agonist or antagonist of CD200 has contacted a cell expressing CD200R.

“Therapeutically effective amount” of a therapeutic agent is defined as an amount of each active component of the pharmaceutical formulation that is sufficient to show a meaningful patient benefit, i.e., to cause a decrease in or amelioration of the symptoms of the condition being treated. When the pharmaceutical formulation comprises a diagnostic agent, “a therapeutically effective amount” is defined as an amount that is sufficient to produce a signal, image, or other diagnostic parameter that facilitates diagnosis. Effective amounts of the pharmaceutical formulation will vary according to factors such as the degree of susceptibility of the individual, the age, gender, and weight of the individual, and idiosyncratic responses of the individual (see, e.g., U.S. Pat. No. 5,888,530).

II. General.

Mammalian skin consists of dermal (inner) and epidermal (outer) layers. The epidermis is made almost entirely of keratinocytes (95%) with other cell types including Langerhans cells and melanocytes. The epidermis is rapidly growing, turning over every seven days in the mouse. Stem cells in the skin divide to produce “transit amplifying cells” which, in turn, divide 3-5 times more, with eventual production of terminally differentiated cells.

The surface of the skin contains two regions, hair follicles and regions between the hair follicles, i.e., interfollicular epidermis. The hair follicle comprises a hair shaft surrounded by layers of epithelial cells that form an inner root sheath and an outer root sheath. For each hair follicle, three cyclic stages of growth and shedding are repeated indefinitely: growth (anagen); regression (catagen); and rest (telogen). In catagen, for example, keratinocytes in the lower region of the follicle are destroyed, where destruction is mediated by apoptosis.

Epidermal stem cells are clustered or located in a structure of the hair follicle called a “bulge.” Stem cells in the bulge divide and supply new cells to various parts of the hair follicle, as well as to the interfollicular epidermis. The “bulge” occurs in a non-cycling part of the hair follicle. A number of markers have been associated with epidermal cells during the course of differentiation. For example, as epidermal cells become committed to terminal differentiation, they switch from expression of keratin-5 and keratin-14 to keratin-1 and keratin-10. In the hair follicle, keratin-5 and keratin-14 tend to be associated with the outer root sheath, while keratins-1 and -10 are in the inner root sheath (see, e.g., Janes, et al. (2002) J. Pathol. 197:479-491; Alonso and Fuchs (2003) Proc. Natl. Acad. Sci. USA 100:11830-11835; Braun, et al. (2003) Development 130:5241-5255; Muller-Rover, et al. (2001) J. Invest. Dermatol. 117:3-15; Niemann and Watt (2002) TRENDS Cell Biol. 12:185-192; Byrne, et al. (1994) Development 120:2369-2383; Vasioukhin, et al. (1999) Proc. Natl. Acad. Sci. USA 96:8551-8556).

Alopecia encompasses hair loss or baldness. The alopecias have been classified as non-scarring alopecias and scarring alopecias, where each form of alopecia is characterized by specific histological features, by the appearance of hair loss, i.e., shape and location of bald spots, and by the affected racial, gender, and age groups. Both types of alopecia have an immunological component, e.g., inflammation, consistent with the inflammation demonstrated in the present invention. Scarring and non-scarring alopecias involve fibrosis, consistent with the fibrosis of the dermal layer found in the observations of the present invention (see, e.g., Zinkernagel, et al. (2000) Arch. Dermatol. 136:205-211; Zinkernagel and Trueb (2000) Arch. Dermatol. 136:205-211; Kossard (1994) Arch. Dermatol. 130:770-774; Whiting (2003) Arch. Dermatol. 139:1555-1559; Chieregato, et al. (2003) Int. J. Dermatol. 42:342-345).

The experiments below show apoptosis of keratinocytes associated with hair follicles. Apoptosis has been documented in scarring alopecia and in non-scarring alopecia. For example, alopecia areata involves apoptosis, as well as cell degeneration, including “dark cell” transformation, and necrosis. The disorder also results in decreased numbers of hair follicles, fibrosis, and a decrease in number of hair follicles in the actively growing phase (anagen phase). Apoptosis has also been documented in, e.g., androgenetic alopecia, pseudopelade, and frontal fibrosing alopecia (see, e.g., Tobin (1997) Microsc. Res. Tech. 15:443-451; Tobin et al. (1991) Am J Dermatopathol. 13:248-56; Bergfeld (1989) Adv. Dermatol. 4:301-320; Trueb and Torricelli (1998) Hautarzt 49:388-391; Morgan and Rose (2003) Ann. Clin. Lab Sci. 33:107-112; Pierard-Franchimont and Pierard (1986) Dermatologica 172:254-257).

The present invention provides methods to treat disorders resulting from changes or breakdown in immune privilege of the hair follicle. Alopecia areata, for example, involves breakdown of hair follicle immune privilege (see, e.g., Perret, et al. (1984) Acta Derm. Venereol. 64:26-30; Ranki, et al. (1984) J. Invest. Dermatol. 83:7-11; Billingham (1971) Adv. Biol. Skin 11:183-198; Billingham and Silvers (1971) J. Invest. Dermatol. 57:227-240; Claesson and Hardt (1970) Transplantation 10:349-351; Paus, et al. (2003) Br. J. Dermatol. 131:177-183; Harrist, et al. (1983) Br. J. Dermatol. 109:623-633; Christoph, et al. (2000) Br. J. Dermatol. 142:862-873; Welker, et al. (1997) Arch. Dermatol. Res. 289:554-557; Slominski, et al. (1998) Biochim. Biophys. Acta 1448:147-152; Botchkarer, et al. (1999) Ann. N.Y. Acad. Sci. 885:433-439; Fuzzi, et al. (2002) Eur. J. Immunol. 32:311-315; Safavi, et al. (1995) Mayo Clin. Proc. 70:628-633; Eichmuller, et al. (1998) J. Histochem. Cytochem. 46:361-370).

III. Binding Compositions.

Binding compositions provided by the methods of the present invention include reagents such as CD200, CD200 receptor (a.k.a. CD200R), a soluble receptor, and antibodies, as well as nucleic acids encoding these reagents. CD200 and CD200 receptor are membrane-bound proteins. CD200 has a broad tissue distribution, while CD200R is expressed, e.g., on myeloid cells. Cell signaling mediated by CD200 and CD200R results in inhibition of immune cell activity. Stimulation of the CD200/CD200R signaling pathway, e.g., with soluble versions of CD200 or with an agonistic anti-CD200R antibody, is effective in treating animal models of various inflammatory disorders. Consistent with this is that inhibition of the CD200/CD200R signaling pathway, e.g., by treating with a blocking anti-CD200 antibody or by the CD200 knockout (CD200KO) technique, accelerates or increases susceptibility to inflammatory disorders. These inflammatory disorders include experimental autoimmune encephalomyelitis (EAE), microglia-mediated nerve damage, collagen-induced arthritis (CIA), transplant rejection, and graft rejection. Moreover, treatment with an antagonist of CD200/CD200R signaling results in increased immune response against tumor cells (see, e.g., Hoek, et al. (2000) Science 290:1768-1771; Gorczynski, et al. (2002) Clin. Immunol. 104:256-264; Gorczynski (2001) Eur. J. Immunol. 31:2331-2337; Gorczynski, et al. (2001) Clin. Exp. Immunol. 126:220-229; Barclay, et al. (2002) TRENDS Immunol. 23:285-290).

CD200 is widely distributed and is expressed, e.g., by T cells, B cells, dendritic cells, neurons, vascular endothelium, kidney glomeruli, corpora lutea, trophoblasts, and smooth muscle. CD200R is more narrowly distributed and is found, e.g., on granulocytes, monocytes, T cells, B cells, NK cells, NKT cells, neutrophils, basophils, and monocytes. Leukemic cells have also been shown to express CD200 (see, e.g., Gorczynski, et al. (2001) Clin. Exp. Immunol. 126:220-229). CD200R occurs as a family of genes, both in rodents and in humans. CD200R of mice occurs as mCD200R, but also as a group of related CD200R-like proteins (CD200RL), named mCD200RLa, mCD200RLb, mCD200RLc, and mCD200RLd. mCD200RLa and mCD200RLb each pair with DAP12, and deliver an activating signal, not an inhibiting signal. mCD200RLa and mCD200RLb appear not to bind to CD200. Human CD200R occurs as hCD200R and hCD200RLa, though hCD200RLa appears not to be expressed (Wright, et al. (2001) Immunology 102:173-179; Wright, et al. (2003) J. Immunol. 171:3034-3046; Lanier and Bakker (2000) Immunol. Today 21:611-614).

The methods of the present invention provide blocking antibodies to CD200, blocking antibodies to CD200R, agonistic antibodies to CD200R, polypeptides derived from the extracellular domains of CD200 or CD200R, e.g., in the form of a soluble receptor, polypeptides derived from the extracellular domain of CD200R, e.g., in the form of a soluble receptor, and fusion proteins of these extracellular domains. The fusion protein comprising two extracellular domains of CD200 and an Fc fragment is known as “CD200 Fc fusion protein,” “CD200Fc,” “CD200-Ig,” “CD200 Ig fusion protein,” and “immunoadhesin.” The Ig fusion protein may contain a mutation (D265A in the constant regions of the Fc) to prevent binding to Fc receptor (FcR) and to complement (see, e.g., Idusogie, et al. (2000) J. Immunol. 164:4178-4184; Wright, et al. (2003) J. Immunol. 171:3034-3046; Gorczynski, et al. (2002) Clin. Immunol. 104:256-264; Chen, et al. (1997) Biochim. Biophs. Acta 1362:6-10).

The extracellular region of mature human CD200 is expected to correspond to about amino acids 31-232 of GenBank NP₁₃ 005935 (gi: 15451904) (see also Chen, et al. (1997) Biochim. Biophys. Acta 1362:6-10). The extracellular region of mature human CD200R is expected to correspond to about amino acids 27 to 242 of GenBank Q8TD46 (gi:26006823) (see also, Wright, et al. (2003) J. Immunol. 171:3034-3046). General methods relating to soluble receptors are available (see, e.g., Monahan, et al. (1997) J. Immunol. 159:4024-4034; Moreland, et al. (1997) New Engl. J. Med. 337:141-147; Borish, et al. (1999) Am. J. Respir. Crit. Care Med. 160:1816-1823; Uchibayashi, et al. (1989) J. Immunol. 142:3901-3908). Provided is a soluble polypeptide of CD200 comprising, e.g., amino acids 31-230; 31-231; 31-232; 31-233; 31-234; and 31-235, of GenBank NP₁₃ 005935. Provided is a soluble polypeptide of CD200R comprising, e.g., amino acids 27-262; 27-263; 27-264; 27-265; 27-266; and 27-267, of GenBank NP₁₃ Q8TD46.

Regions of increased antigenicity of human CD200 occur, e.g., at amino acids 36-42; 54-59; 65-74; 79-83; 87-93; 111-118; 159-168; 175-197; 202-211; and 260-268, of GenBank NP₁₃ 005935, while regions of increased antigenicity of human CD200R occur, e.g., at amino acids 29-40; 79-104; 109-116; 136-140; 159-178; 182-191; 194-204; 235-242; 266-281; 284-300; and 303-313, of GenBank Q8TD46, according to Parker plot analysis using Vector NTI Suite 7® (Accelrys, San Diego, Calif.). Intact protein, denatured protein, or a free or conjugated peptide fragment of the protein, may be used for immunization (see, e.g., Harlow and Lane (1988) Antibodies A Laboratory Manual, Cold Spring Harbor Laboratory Press, Cold Spring Harbor, N.Y., pp. 139-243).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378; Baca, et al. (1997) J. Biol. Chem. 272:10678-10684; Chothia, et al. (1989) Nature 342:877-883; Foote and Winter (1992) J. Mol. Biol. 224:487-499; U.S. Pat. No. 6,329,511 issued to Vasquez, et al.).

An alternative to humanization is to use human antibody libraries displayed on phage or human antibody libraries in transgenic mice (Vaughan, et al. (1996) Nature Biotechnol. 14:309-314; Barbas (1995) Nature Medicine 1:837-839; Mendez, et al. (1997) Nature Genetics 15:146-156; Hoogenboom and Chames (2000) Immunol Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nature Biotechnol. 17:397-399).

Single chain antibodies and diabodies are described (see, e.g., Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914).

The present invention provides a bispecific antibody that can bind specifically CD200R (an inhibiting receptor) and an activating receptor, including an activating receptor that is an ITAM containing receptor. Simultaneous binding of the bifunctional antibody to CD200R and to an activating receptor results in cross-linking of CD200R and the activating receptor. For example, the present invention provides a bispecific antibody that binds CD200R and a polypeptide of T cell receptor; a bispecific antibody that binds CD200R and FcepsilonRI; and a bispecific antibody that binds CD200R and FcgammaRIIA. The consensus ITAM sequence is YxxL/Ix₆₋₈YxxL/I, where (Y) may be phosphorylated resulting in a change in signaling properties of the activating receptor and/or the accessory protein. The ITAM motif may occur within an activating receptor itself, or within an accessory protein that binds to the activating receptor, thus conferring activating properties to the activating receptor. Activating receptors, including ITAM-motif containing receptors, include e.g., CD3, CD2, CD10, CD161, DAP-12, KAR, KARAP, FcepsilonRI, FcepsilonRII, FcgammaRIIA, FcgammaRIIC, FcgammaRIII/CD16, Trem-1, Trem-2, CD28, p44, p46, B cell receptor, LMP2A, STAM, STAM-2, GPVI, and CD40 (see, e.g., Azzoni, et al. (1998) J. Immunol 161:3493; Kita, et al. (1999) J. Immunol. 162:6901; Merchant, et al. (2000) J. Biol. Chem. 74:9115; Pandey, et al. (2000) J. Biol. Chem. 275:38633; Zheng, et al. (2001) J. Biol Chem. 276:12999; Propst, et al. (2000) J. Immunol. 165:2214; Long (1999) Ann. Rev. Immunol. 17:875).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard, et al. (1997) Immunity 7:283-290; Wright, et al. (2000) Immunity 13:233-242; Preston, et al., supra; Kaithamana, et al. (1999) J. Immunol. 163:5157-5164).

Antibodies will usually bind with at least a K_{D} of about 10^{31 3} M, more usually at least 10^{31 6} M, typically at least 10^{31 7} M, more typically at least 10^{31 8} M, preferably at least about 10^{31 9} M, and more preferably at least 10^{31 10} M, and most preferably at least 10^{31 11} M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

Polypeptides, antibodies, and nucleic acids, can be conjugated, e.g., to small drug molecules, enzymes, liposomes, polyethylene glycol (PEG), or fusion protein antibodies. Antibodies are useful for diagnostic or kit purposes, and include antibodies coupled, e.g., to dyes, radioisotopes, enzymes, or metals, e.g., colloidal gold (see, e.g., Le Doussal, et al. (1991) J. Immunol. 146:169-175; Gibellini, et al. (1998) J. Immunol. 160:3891-3898; Hsing and Bishop (1999) J. Immunol. 162:2804-2811; Everts, et al. (2002) J. Immunol. 168:883-889).

The invention also provides binding compositions for use as anti-sense nucleic acids or for small interference RNA (siRNA) (see, e.g., Arenz and Schepers (2003) Naturwissenschaften 90:345-359; Sazani and Kole (2003) J. Clin. Invest. 112:481486; Pirollo, et al. (2003) Pharmacol. Therapeutics 99:55-77; Wang, et al. (2003) Antisense Nucl. Acid Drug Devel. 13:169-189; Cheng, et al. (2003) Mol. Genet. Metab. 80:121-128; Kittler and Buchholz (2003) Semin. Cancer Biol. 13:259-265).

The invention encompasses methods of using a reagent to increase expression of CD200 or of CD200R. Agents that increase expression of receptors on a cell surface are useful for increasing the effective concentration of target receptors on the cell surface, thus increasing the activity of a binding composition specific for that receptor (see, e.g., van de Winkel, et al. (1991) J. Leukocyte Biol. 49:511-524; van de Winkel, et al. (1993) Immunol. Today 14:215-221; Heijnen, et al. (1997) Intern. Rev. Immunol. 16:29-55; Fridman and Sautes (1996) Cell-Mediated Effects of Immunoglobins, Chapman and Hall, New York, N.Y., pp. 3940).

IV. Purification and Modification of Polypeptides and Nucleic Acids.

Polypeptides, e.g., antigens, antibodies, and antibody fragments, and nucleic acids for use in the contemplated method, can be purified by methods that are established in the art. Purification can involve homogenization of cells or tissues, immunoprecipitation, and chromatography. Stability during purification or storage can be enhanced, e.g., by anti-protease agents, anti-oxidants, ionic and non-ionic detergents, and solvents, such as glycerol or dimethylsulfoxide.

Modification of, e.g., peptides, polypeptides, and nucleic acids, includes epitope tags, fluorescent or radioactive groups, monosaccharides or oligosaccharides, sulfate or phosphate groups, C-terminal amides, acetylated and esterified N-groups, acylation, e.g., fatty acid, intrachain cleaved peptide bonds, and deamidation products (see, e.g., Johnson, et al. (1989) J. Biol. Chem. 264:14262-14271; Young, et al. (2001) J. Biol. Chem. 276:37161-37165). Glycosylation depends upon the nature of the recombinant host organism employed or physiological state (see, e.g., Jefferis (2001) BioPharm 14:19-27; Mimura, et al. (2001) J. Biol. Chem. 276:45539-45547; Axford (1999) Biochim. Biophys. Acta 1:219-229; Malhotra, et al. (1995) Nature Medicine 1:237-243).

V. Therapeutic Compositions and Methods.

To prepare pharmaceutical or sterile compositions including an agonist or antagonist of CD200 or CD200R, the reagents is mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg body weight basis. The desired plasma concentration of a small molecule therapeutic is about the same as for an antibody, on a moles/kg body weight basis.

An effective amount for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK.

Typical veterinary, experimental, or research subjects include monkeys, dogs, cats, rats, mice, rabbits, guinea pigs, horses, and humans.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

Methods for co-administration or treatment with a second therapeutic agent, e.g., a cytokine, steroid, chemotherapeutic agent, antibiotic, or radiation, are well known in the art (see, e.g., Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic will decrease the symptoms typically by at least 10%; usually by at least 20%; preferably at least about 30%; more preferably at least 40%, and most preferably by at least 50%.

The route of administration is by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad. Sci. USA 82:3688-3692; Hwang, et al. (1980) Proc. Natl. Acad. Sci. USA 77:4030-4034; U.S. Pat. Nos. 6,350,466 and 6,316,024).

VI. Kits.

The present invention provides methods of using agonists and antagonist of CD200, e.g., proteins, fragments thereof, binding compositions derived from an antibody, nucleic acids, and fragments thereof, in a diagnostic kit. Also provided are binding compositions, including antibodies or antibody fragments, for the detection of CD200 or CD200R, and metabolites and breakdown products thereof, including products resulting from deamidation, limited proteolytic or hydrolytic cleavage, or disulfide bond oxidation or formation. Typically, the kit will have a compartment containing either a CD200 or CD200R polypeptide, or an antigenic fragment thereof, a binding composition thereto, or a nucleic acid, e.g., a nucleic acid probe or primer, able to hybridize under stringent conditions to a nucleic acid encoding CD200 or CD200R.

The kit can comprise, e.g., a reagent and a compartment, a reagent and instructions for use, or a reagent with a compartment and instructions for use. The reagent can comprise a CD200, CD200R, or soluble version derived from the extracellular region, or an antigenic fragment thereof, a binding composition, or a nucleic acid. A kit for determining the binding of a test compound, e.g., acquired from a biological sample or from a chemical library, can comprise a control compound, a labeled compound, and a method for separating free labeled compound from bound labeled compound.

Conditions enabling stringent hybridization of nucleic acid probes or primers are available (see, e.g., Freeman, et al. (2000) Biotechniques 29:1042-1055; de Silva and Wittwer (2000) J. Chromatogr. B. Biomed. Sci. Appl. 741:3-13; Long (1998) Eur. J. Histochem. 42:101-109; Musiani, et al. (1998) Histol. Histopathol. 13:243-248; Gillespie (1990) Vet. Microbiol. 24:217-233; Giulietti, et al. (2001) Methods 25:386-401; Schweitzer and Kingsmore (2001) Curr. Opin. Biotechnol. 12:21-27; Speel, et al. (1999) J. Histochem. Cytochem. 47:281-288; Tsuruoka and Karube (2003) Comb. Chem. High Throughput Screen. 6:225-234; Rose, et al. (2002) Biotechniques 33:54-56).

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes, enzymes, and metals (see, e.g., Le Doussal, et al. (1991) New Engl. J. Med. 146:169-175; Gibellini, et al. (1998) J. Immunol. 160:3891-3898; Hsing and Bishop (1999) New Engl. J. Med. 162:2804-2811; Everts, et al. (2002) New Engl. J. Med. 168:883-889). Various assay formats exist, such as radioimmunoassays (RIA), ELISA, and lab on a chip (U.S. Pat. Nos. 6,176,962 and 6,517,234).

The diagnostic method can comprise contacting a sample from a test subject with a binding composition that specifically binds to a polypeptide or nucleic acid of CD200 or CD200R. Moreover, the diagnostic method can further comprise contacting the binding composition to a sample derived from a control subject or control sample, and comparing the binding found with the test subject with the binding found with the control subject or control sample. A “test sample” can be derived from a skin sample from a subject experiencing alopecia, while a “control sample” can be derived from a skin sample from a normal subject, or derived from a non-affected skin sample from the subject experiencing alopecia. The subject can be, e.g., human, veterinary, experimental, or agricultural. Derived encompasses a biopsy, sample, extract, or a processed, purified, or semi-purified sample or extract.

VII. Uses.

The invention provides methods for the diagnosis, treatment, or prevention of disorders of the hair follicle, including proliferative disorders and inflammatory disorders of the hair follicle, e.g., scarring and non-scarring alopecia. Provided are methods for treating androgenetic alopecia (AGA), alopecia areata (AA), and traction alopecia. Also provided are methods for treating pseudopelade of Brocq (PB), chronic, cutaneous lupus erythematosus (CCLE), lichen planopilaris (LPP), dissecting cellulites, acne keloidalis, central, centrifugal scarring alopecia (CCSA), and fibrosing alopecia. Moreover, the present invention provides methods for the treatment and diagnosis of loose anagen syndrome, chronic telogen effluvium, and the frontal fibrosing variant of lichen planopilaris. Provided are methods of treatment and diagnosis of hair loss and baldness, including drug induced hair loss (see, e.g., Tosi, et al. (1994) Drug Saf. 10:310-317; Sullivan and Kossard (1998) Australas J. Dermatol. 39:207-218).

Also provided are methods for treating or diagnosing inflammatory disorders or autoimmune disorders of immune privileged regions of the body. Immune privileged regions of the body include the hair follicle, eye, central nervous system, brain, and reproductive system (Christoph, et al. (2000) Br. J. Dermatol. 142:862-873; Streilein and Stein-Streilein (2000) J. Leukocyte Biol. 67:479-487; Ferguson, et al. (2002) Int. Rev. Immunol. 21:153-172; Paus, et al. (2003) J. Investig. Dermatol. Symp. Proc. 8:188-194).

Moreover, the present invention provides methods of using a depilatory agent. The depilatory agent comprises, e.g., an antagonist of CD200, for example, an anti-CD200 antibody, a blocking anti-CD200R antibody, a soluble version of the extracellular region of CD200R, or a peptide mimetic thereof. Present methods of hair removal are not completely satisfactory and lead to side effects, e.g., hypo- and hyperpigmentation (Topping, et al. (2000) Ann. Plast. Surg. 44:668-674; Liew (1999) Dermatol. Surg. 25:431439; Olsen (1999) J. Am. Acad. Dermatol. 40:143-155; de Berker (1999) Practitioner 243:493-498; Lanigan (2001) Clin. Exp. Dermatol. 26:644-647; Liew (2002) Am. J. Clin. Dermatol. 3:107-115; Trueb (2002) Am. J. Clin. Dermatol. 3:617-627).

The depilatory agent can be used in conjunction with an inflammatory agent or an immune activating agent, e.g., an inflammatory cytokine, a TH1-type cytokine, a TH2-type cytokine, a skin irritant, or an agent that stimulates contact hypersensitivity or dermatitis (see, e.g., Chew and Maibach (2003) Int. Arch. Occup. Environ. Health 76:339-346; Antexana and Parker (2003) Immunol. Allergy Clin. North Am. 23:269-290; Willis (2002) Contact Dermatitis 47:267-271; Smith, et al. (2002) Clin. Exp. Dermatol. 27:138-146; Wollenberg and Bieber (2001) Transplant Proc. 33:2212-2216).

The broad scope of this invention is best understood with reference to the following examples, which are not intended to limit the inventions to the specific embodiments.

### EXAMPLES

I. General Methods.

Methods for the diagnosis and treatment of inflammatory conditions of the skin in animals and humans are described (see, e.g., Ackerman (1997) Histological Diagnosis of Inflammatory Skin Disease, 2^{nd} ed., Lippincott, Williams, and Wilkins, New York, N.Y.; Gallin, et al. (1999) Inflammation: Basic Principles and Clinical Correlates, 3^{rd} ed., Lippincott, Williams, and Wilkins, New York, N.Y.; Parnham, et al. (1991) Drugs in Inflammation (Agents and Actions Suppl., Vol. 32), Springer Verlag, Inc., New York, N.Y.; Chan (ed.) (2003) Animal Models of Human Inflammatory Skin Diseases, CRC Press, Boca Raton, Fla.; Kownatzki and Norgauer (eds.) (1998) Chemikines and Skin, Birkhauser Verlag, Basel, Switzerland; Kanitakis, et al. (eds.) (1999) Diagnostic Immunohistochemistry of the Skin, Lippincott, Williams, and Wilkins, New York, N.Y.).

Animal models of alopecia, and related methods, are available. These methods include use of skin grafts, skin grafts injected with immune cells, subcutaneous injection of immune cells, and use of animals such as the Dundee experimental bald rat (see, e.g., Zoller, et al. (2002) J. Invest. Dermatol. 118:983-992; Sundberg, et al. (2001) Eur. J. Dermatol. 11:321-325; Sundberg, et al. (2000) Am. J. Pathol. 156:2067-2075; McElwee and Hoffmann (2002) Clin. Exp. Dermatol. 27:410-417; McElwee, et al. (1996) Br. J. Dermatol. 135:211-217; McElwee, et al. (1996) Br. J. Dermatol. 135:211-217).

Methods for the classification of human and animal hair follicles are available (see, e.g., Muller-Rover, et al. (2001) J. Invest. Dermatol. 117:3-15; Millar (2002) J. Invest. Dermatol. 118:216-225). General methods of skin pathology and dermatology are available (see, e.g., Bos (ed.) (1997) The Skin Immune System, CRC Press, Boca Raton, Fla.; Weedon (2002) Skin Pathology, 2^{nd} ed., Churchill Livingston, Phila., Pa.; Hobif, et al. (eds.) (2001) Skin Disease: Diagnosis and Treatment, Mosby, Phila., Pa.; Habif and Habie (1996) Clinical Dermatology, 4^{th} ed., Mosby, Phila., Pa.; Muller, et al. (2000) Muller and Kirk's Small Animal Dermatology, 6^{th}ed., W. B. Saunders, Phila., Pa.; Weston, et al. (2002) Color Textbook of Pediatric Dermatology, 3^{rd} ed., Mosby, Phila., Pa.).

Standard methods in molecular biology are described (Maniatis, et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies is described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protcols in Immunology, Vol. 4, John Wiley, Inc., New York).

Standard techniques in cell and tissue culture are described (see, e.g., Freshney (2000) Culture of Animal Cells: A Manual of Basic Technique, 4^{th} ed., Wiley-Liss, Hoboken, N.J.; Masters (ed.) (2000) Animal Cell Culture: A Practical Approach, 3^{rd} ed., Oxford Univ. Press, Oxford, UK; Doyle, et al. (eds.) (1994) Cell and Tissue Culture: Laboratory Procedures, John Wiley and Sons, N.Y.; Melamed, et al. (1990) Flow Cytometry and Sorting Wiley-Liss, Inc., New York, N.Y.; Shapiro (1988) Practical Flow Cytometry Liss, New York, N.Y.; Robinson, et al. (1993) Handbook of Flow Cytometry Methods, Wiley-Liss, New York, N.Y.).

Software packages for determining, e.g., antigenic fragments, signal and leader sequences, protein folding, and functional domains, are available. See, e.g., Vector NTI® Suite (Informax, Inc., Bethesda, Md.); GCG Wisconsin Package (Accelrys, Inc., San Diego, Calif.), and DeCypher® (TimeLogic Corp.; Crystal Bay, Nev.); Menne, et al. (2000) Bioinformatics 16:741-742. Public sequence databases were also used, e.g., from GenBank and others.

II. Methods for Keratinocyte Culture, Histology, and Skin Grafting.

C57BL/6 mice (B6) were obtained from Jackson Laboratories (Bar Harbor, Me.). CD200KO mice were derived from a B6 background (DNAX Research, Inc., Palo Alto, Calif.). Age/sex matched mice were used in all experiments. The murine KC cell lines, PAM212, SP-1, and 308 were from Stuart Yuspa (National Institute of Health, Bethesda, Md.).

Cell lines were cultured in Dulbecco's modified Eagle medium (DMEM) (GIBCO BRL, Grand Island, N.Y.) supplemented with 10% fetal bovine serum (FBS). Human keratinocytes were derived from newborn human foreskins and cultured in Keratinocyte SFM (GIBCO BRL; Rheinwald and Green Cell 6:317-330). Neonatal trunk or adult ear skin was excised from B6 mice and epidermal cells (ECs) Tamaki, et al. (1979) J. Immunol. 123:784-787. Skin was separated by gently tearing along the cartilage plate and floated on 0.5% trypsin (GIBCO BRL) in phosphate buffered saline (PBS) at 37° C. for 45 min. Epidermal sheets were peeled from the dermis, re-suspended in 0.05% DNAase (Sigma, St. Louis, Mo.) in PBS containing 10% fetal bovine serum (FBS). Single cell suspension was obtained by vigorous passage through a syringe. For reverse transcription polymerase chain reaction (RT-PCR) analysis, cells were cultured in Keratinocyte SFM.

For flow cytometry, freshly isolated epidermal cells were washed once in cold phosphate buffered saline (PBS) and 4×10⁵ cells were stained for 30 min at 4° C. with any of the following reagents: Alexa Fluor-647 (Molecular Probes, Eugene, Oreg.) conjugated anti-mCD200 antibody (OX-90); Alexa Fluor-647 conjugated rat IgG isotype control (R35-95); PE anti-hCD200 (MRC OX-104); FITC anti-I-A^{b} (KH74); PE anti-CD3 (145-2C11); 7AAD (CalBiochem, La Jolla, Calif.). Antibodies were from Pharmingen (San Diego, Calif.). OX-90 and R35-95 mAbs were conjugated to Alexa Fluor 647 according to manufacturer's protocol. Cells were washed twice in cold PBS and analyzed by flow cytometry on a Becton Dickenson FACScan® flow cytometer (San Jose, Calif.).

Keratinocytes were isolated as described above and cultured in Keratinocyte SFM. After two passages, cells were harvested, and total RNA was extracted with TRIzol® (Life Technologies, Rockville, Md.). RNA was quantified, and equal amounts (about one microgram) were reverse transcribed into cDNA with oligo(dT) primers using Thermoscripto ® RT-PCR systems (Gibco BRL, Grand Island, N.Y.). RT-PCR was performed using primers hybridizing to the following regions of mouse CD200, one 20-base primer hybridizing to nucleotides 123 to 141, and a second 19-base primer hybridizing to nucleotides 441 to 458 of the nucleic acid sequence of GenBank NM₁₃ 010818. A second primer set was used for assessing expression of beta-actin.

Neonatal trunk skin was isolated from either wild type (WT) or CD200KO mice. Specimens were immediately placed in Tissue-Tek OCT Compound (Miles Inc., Elkhart, Ind.), frozen on dry ice, and stored at −70° C. Cryosections (6 micrometers) were stained for immunofluorescence microscopy (Basset-Seguin, et al. (1988) J. Immunol. 141:1273-1280). Anti-mCD200 (OX-90) or rat IgG isotype control (R35-95) were used as primary antibodies and FITC-conjugated goat F(ab)₂ anti-rat IgG (Jackson lmmunoresearch Laboratories, West Grove, Pa.) was used for detection. Immunohistochemistry was carried out on frozen sections as described (Homey, et al. (2000) J. Immunol. 164:6621-6632). Anti-mCD200 mAb or rat IgG isotype control mAb binding was detected using biotinylated rabbit anti-rat IgG (Vector Biosys, Compiegne, France) followed by streptavidin-peroxidase. The reagents were from Vectastain ABC kit, Vector Biosys. Peroxidase activity was revealed using 3-amino-9-ethylcarbazole substrate (SK-4200, Vector) for 5-10 min at room temperature.

Tail skin was grafted to the dorsal trunk as described (Coligan, et al. (eds.) (1994) Skin Allograft Rejection in Current Protocols in Immunology, John Wiley, New York). Briefly, tail skin was harvested from age-matched wild type and CD200KO female B6 mice and grafted onto the backs of age-matched wild type B6 females. In some experiments, wild type and CD200KO skin was grafted onto the same host, and in others, each host received only one graft. Skin was observed daily, and at various times post-grafting, punch biopsies were taken. Specimens were fixed in 4% fomalin in PBS, embedded in paraffin, sectioned at 5 micrometers thickness, and stained with hematoxylineosin (H&E).

III. Expression of CD200 by Keratinocytes and Hair Follicles.

CD200-specific RT-PCR was performed on both mouse and human primary keratinocyte cell (KC) cultures, as follows. Epidermal cells (ECs) were isolated from both mouse pup skin and human foreskin and cultured in KC-defined media. CD200 mRNA was detected in both mouse and human primary KC cultures. As a control, RT-PCR was also performed on splenocyte mRNA isolated from wild type mice or CD200KO B6 mice. Expression by wild type splenocytes was somewhat less than from mouse or human keratinocytes, while expression by splenocytes from CD200KO mice was absent.

To determine if keratinocytes express CD200 on their cell surface, 4-color flow cytometry was performed on freshly isolated epidermal cells derived from mouse pup skin. After isolation from wild type and from CD200KO mice, epidermal cells were separated by FACS analysis into three different EC populations. These three cell populations were: (1) T cells (CD3⁺, MHC II⁻): (2) Langerhans cells (CD3⁻, MHC II⁺); and (3) Keratinocytes (CD3⁻, MHC II⁻). The three cell populations were separated from each other by a FACS machine, and cells isolated from wild type mice were analyzed for CD200 expression. The three cell populations from CD200KO mice were analyzed as a control (Table 1).

TABLE 1
Expression of CD200 by subpopulations of epidermal cells freshly
isolated from mouse pup skin.

| | | | Expression of CD200 | |
|---|---|---|---|---|
| | | | by FACS analysis | |
| | Phenotype | | Percent | Percent |
| Cell type | CD3 | MHCII | CD200^{hi} | CD200^{low or neg.} |
| Langerhans | minus | high | 44% | 56% |
| cells | | | | |
| T cells | high | minus | 2% | 98% |
| Keratinocytes | minus | minus | 15% | 85% |

Langerhans cells from wild type mice showed expression of CD200, where expression was found in roughly 44% of the cells. Keratinocytes from wild type mice showed a biphasic distribution, that is, two distinct populations, where 15% showed expression of CD200, and 85% showed little or no expression of CD200. T cells from wild type mice showed little or no expression (2% of cells) of CD200 (Table 1). As expected, cells prepared from CD200KO mice showed little or no signal for CD200 (Langerhans cells at 5%, T cells at 2%, and KCs at 1%).

When adult mouse ear skin was used as the source of epidermal cells, a somewhat lower percentage of CD200⁺ cells in the CD3⁻ MHC II⁻ cell population was found (between 5-15%), relative to that found in cells from mouse pup skin (about 15%).

To determine if cultured keratinocytes (MHC II⁻; CD3⁻ phenotype) express cell surface CD200, several murine keratinocyte cell lines as well as primary human keratinocyte cultures were analyzed by flow cytometry. Primary cultures of human keratinocytes and the murine keratinocyte cell lines PAM212 and SP-1 did not express CD200.

Scanning confocal microscopy of mouse epidermis demonstrated that a subpopulation of MHC Class II negative, CD3 negative cells expressed CD200, but also expressed keratin-14. This staining was accentuated in keratinocytes of the hair follicle outer root sheath. The phenotype of co-expression with keratin-14 indicated that the cell was a stem cell or a transit amplifying cell.

Localization of CD200-expressing cells in the epidermis was determined using biopsies of neonatal trunk skin. Cell location was determined by CD200-specific immunofluorescence and CD200-specific immunohistochemistry on whole mounts of mouse pup skin. CD200 expression was localized almost exclusively to hair follicles. Specific CD200 staining was not observed in non-hair follicle associated epidermis. CD200⁺ cells were located primarily in the outer root sheath of the hair follicle, with relatively uniform expression throughout the length of the follicle. CD200 expression was observed surrounding the bulb, isthmus, bulge and infundibular regions. Bulb matrix cells, dermal papillary cells, and cell of the hair shaft did not appear to express CD200. A similar pattern of expression was observed in adult ear skin. As a control, CD200 staining was also observed on vascular endothelial cells, as has been previously reported (Clark, et al. (1985) EMBO J. 4:113-118).

IV. CD200 Suppresses Hair Follicle-Associated Autoimmunity.

The absence of CD200 accelerated the rejection of skin grafts or rejection of hair follicles only, as shown after grafting donor skin to female mice recipients. Tail skin from a donor mouse was grafted to the trunk of female wild type recipient mice. At various times after grafting, punch biopsies were formed, and sections were stained with hematoxylin-eosin. Syngeneic skin grafting model in which tail skin from either male or female wild type (CD200^{+/+}) or CD200KO B6 mice (CD200^{-/-} B6 mice) were grafted onto the backs of wild type female B6 recipients.

Female to female grafts were studied. An increased inflammatory cell infiltrate was observed in the dermis of female CD200KO grafts relative to wild type female grafts as early as 10 days post-transplant. The infiltrate consisted of polymorphonuclear cells as well as mononuclear cells and was localized in perifollicular and intrafollicular regions of hair follicles. This infiltration was not observed in wild type grafts. At 40 days post-transplant, normal hair follicle architecture in CD200KO grafts was replaced by inflammatory cells, accompanied by intrafollicular edema and intrafollicular apoptosis. Inflammatory cells were rarely observed in the interfollicular dermis and non-hair follicle associated epidermis. By 80 days post-transplant, the hair on 11 out of 11 CD200KO female grafts was completely lost, while the skin graft itself remained intact.

Further details of female to female grafts were as follows. Both scarring and non-scarring outcomes were found. Histological examination confirmed complete loss of hair follicle structures. In some CD200KO grafts, dermal inflammation resolved after hair follicle loss leaving behind dermal scarring. In these grafts, non-hair follicle associated epidermis remained largely unaffected and hairless grafts persisted long-term. However, in some CD200KO grafts inflammation persisted in the dermis after hair follicle elimination with involvement of non-hair follicle associated epidermis, eventually leading to graft lost. In contrast to CD200KO grafts, wild type grafts showed only minimal non-hair follicle associated inflammation early post-transplant 10 days, which resolved entirely by 40 days. No hair follicle loss was observed in wild type grafts and all wild type grafts persisted long-term with hair (over 120 days).

CD200KO mice do have hair, though hair loss occurs with aging, as noted below. The loss of hair follicles (but not of the skin graft) found in transplantation of CD200KO skin from a female donor to a female recipient indicates that the surgical procedure of skin grafting provides an inflammatory trigger, resulting in low levels of inflammation, where this low level of inflammation overwhelms the hair follicles of the CD200^{-/-} skin graft.

The results of male to female grafts were as follows. Male wild type grafts were rejected in approximately one month. Male CD200KO grafts were rejected more rapidly, that is, in about two weeks, demonstrating a role for CD200 in protecting the male skin graft from rejection. Male to female grafts were met with increased graft rejection, as compared to female to female grafts, apparently because of a heightened response to H-Y antigens. H-Y antigens refers to the collection of minor histocompatibility antigens that are encoded by genes on the male (Y) chromosome (see, e.g., James, et al. (2002) Int. Immunol. 14:1333-1342).

Age-associated effects in CD200KO mice were also addressed. Aged CD200KO mice showed alopecia and hair follicle depigmentation. An examination of older sCD200KO mice demonstrated the following. Some of the older CD200KO mice showed signs of hair follicle-associated autoimmunity as evidenced by alopecia and hair shaft depigmentation at about 8 months of age. These age-associated effects were not observed in wild type B6 mice at any age. Wild type C57BL/6J do not normally develop alopecia (see, e.g., Sundberg et al. (2003) Invest. Dermatol. 120:771-775).

The present invention provides methods to modulate T cell activity for the treatment of, e.g., alopecia. CD200 and CD200R signaling controls T cell expression of cytokines. Soluble CD200 contacted to CD200R⁺ T cells resulted in an increase in expression of insulin-like growth factor-1 and of interferon-gamma. These two cytokines regulate hair growth (see, e.g., Signorello, et al. (1999) J. Am. Acad. Dermatol. 40:200-203; Hirota, et al. (2002) J. Interferon Cytokine Res. 22:935-945).

V. CD200 Expression by Keratinocyte-Derived Tumor Cell Lines.

The present invention provides methods to treat keratinocyte-derived tumors and cancers, e.g., by providing an agonist of CD200. A keratinocyte tumor cell line (308 cell line) initiated in vivo with 7, 12-dimethylbenz(a)anthracene, expressed high levels of CD200. 308 cells are described (see, e.g., Strickland, et al. (1988) Cancer Res. 48:165-169). Thus, keratinocyte-derived tumors could utilize CD200 expression to inactivate cells of the immune system, and thus evade anti-tumor immunity. Two other mouse keratinocyte tumor cell lines, PAM212 and SP-1, were found not to express CD200. CD200 expression was also found in other tumor cell lines: C1498 (mouse leukemia), SCC-7 (mouse squamous cell carcinoma), and U2OS (human osteosarcoma).

VI. Expression of CD200R in Murine Epidermis

C57BL/6 mice (B6) were purchased from Jackson Laboratories (Bar Harbor, Me.). CD200^{-/-} mice (derived in the B6 background (Hoek et al., supra) were provided by Dr. Jonathan Sedgwick (DNAX Research Institute, Palo Alto, Calif.)). All mice were housed in the Medical College of Wisconsin's Animal Resource Center, which is accredited by the American Association for the Accreditation of Laboratory Animal Care.

a. Epidermal Cell Preparations

Adult ear skin was excised from B6 mice and ECs were isolated as previously described (Tamaki et al. (1979) J. Immunol. 123:784-787). Briefly, skin was separated from the cartilage plate and floated on 0.5% trypsin (GIBCO BRL) in PBS at 37° C. for 45 min. Epidermal sheets were peeled from the dermis, re-suspended in 0.05% DNAase (Sigma, St Louis, Mo.) in PBS containing 10% FBS. Single cell suspension was obtained by vigorous passage through a 60 cc syringe. For flow cytometry, freshly isolated ECs were washed 1× in cold PBS and 4×10⁵ cells were stained for 30 min. at 4° C. with the following: Alexa Fluor-647 (Molecular Probes, Eugene, Oreg.) conjugated anti-mCD200R1 antibody (OX-110); Alexa Fluor-647 conjugated rat IgG isotype control (R35-95); PE anti-mouse γδ TCR (GL3); FITC anti-I-A^{b} (KH74); 7AAD (CalBiochem, La Jolla, Calif.). All antibodies were from Pharmingen (San Diego, Calif.) except OX-110, which was generously provided by Neil A. Barclay (University of Oxford, UK). OX-110 and R35-95 mAbs were conjugated to Alexa Fluor 647 according to manufacturer's protocol. Cells were washed twice in cold PBS and analyzed by flow cytometry on a Becton Dickenson (San Jose, Calif.) FACScan flow cytometer. For RT-PCR analysis, purified epidermal leukocyte populations were obtained by fluorescent activated cell sorting (FACS). Epidermal cell suspensions were stained with PE anti-mouse γδ TCR, FITC anti-I-A^{b} and 7AAD. Gamma delta TCR⁺/I-A⁻/7AAD⁻ (DETCs), γδ TCR⁻/I-A⁺/7AAD⁻ (LCs), and γδ TCR⁻/I-A⁻/7AAD⁻ (KCs) cells were sorted to ≧99% purity with a BD FACS DiVa with BD TurboSort Plus options. For activation experiments, purified DETCs were cultured in 96-well plates pre-coated with 10 μg/ml anti-CD3 (145-2C11; Pharmingen) at 37° C. in 5% CO₂ for up to 72 hrs. Culture media was RPMI with 10% heat-inactivated fetal bovine serum supplemented with 50 μM 2-mercaptoethanol (Sigma, St Louis, Mo.), HEPES buffer (25 mM), sodium pyruvate (1 mM), penicillin (100 U/ml), streptomycin (100 ug/ml), L-glutamine (2 mM), 100 μM non-essential amino acids, and 20 U/ml recombinant human IL-2. All components were obtained from Gibco BRL (Grand Island, N.Y.) unless otherwise specified.

b. Quantitative RT-PCR

Epidermal cell suspensions were sorted into purified DETC (dendritic epidermal T cells), LC (Langerhans cells), and KC (keratinocytes) populations as described above. Cells were harvested, and total RNA was extracted with TRIzol according to manufacturers instructions (Life Technologies, Rockville, Md.). RNA was quantified, and equal amounts (˜1 μg) were reverse transcribed into cDNA with oligo(dT) primers using Thermoscript™ RT-PCR systems (Gibco BRL, Grand Island, N.Y.) according to manufacturers instructions. To increase the sensitivity of detection in experiments were DETCs were sorted and subsequently activated, 10 ng of total RNA was amplified prior to qRT-PCR using the Ovation™ RNA amplification system according to manufacturer's protocol (NuGen Technologies, San Carlos, Calif.). Quantitative real-time PCR was performed. As control, pre-formulated 18S rRNA Gene Expression Assay systems was utilized according to manufacturer's protocol (Applied Biosystems, Foster City, Calif.). As control, β-actin expression was compared to CD200R isoform expression using SYBR® Green detection reagent according to manufacturer's protocol (Stratagene, La Jolla, Calif.) All qRT-PCR reactions were carried out in an Opticon-2 Continuous Fluorescence Detector (MJ Research, Boston, Mass.). Data was analyzed using the comparative Ct method (Applied Biosystems).

c. Construction of CD200.FLAG and BAP.FLAG Fusion Proteins

Unless noted otherwise, all procedures were done according to the instructions provided by the annotated manufacturer. Total RNA was extracted from murine splenocytes using TRIzol™. RNA was reverse transcribed into cDNA with oligo(dT) primers using Thermoscript™ RT-PCR systems. The following primers specific for the extracellular domain of murine CD200, containing Hind III and Bam H1 restriction sites were synthesized (Invitrogen, Grand Isalnd, N.Y.). Amplified products were then cloned and sequenced utilizing the pCR® 2.1-TOPO cloning vector and the TOPO™ TA Cloning kite® (Invitrogen, Grand Isalnd, N.Y.). Inserts were cloned into the p3XFLAG-CMV™-13 expression vector utilizing Hind3 and BamH1 restriction enzymes (Sigma, St. Louis, Mich.). This vector is designed for the stable expression and secretion of C-terminal-linked 3×FLAG fusion proteins. As control, FLAG tagged Bacterial Alkaline Phosphatase (BAP) fusion protein vector (pFLAG-CMV-3-BAP) was purchased from Sigma. Chinese hamster ovary cells (CHO) were nucleofected with either CD200.FLAG or BAP.FLAG vectors (Amaxa Biosystems, Koeln, Germany). Nucleofection was optimized using Amaxa's Cell Line Optimization Nucleofector Kit. Culture supernatants were harvested 5-7 days later and filtered concentrated using YM-10 Centripluse® centrifugal filter devices (Millipore, Bedford, Mass.). Concentrated fusion protein was assayed for purity by western blot using anti-FLAG ® M2 antibody (Sigma, St. Louis, Mich.). Fusion protein was quantified by densitometry on western blots with known concentrations of 3×FLAG-BAP protein as standard (Sigma, St. Louis, Mich.) using Alpha Imager 2200 v5.5 software on an Alpha Imager 2200 (Alpha Innotech Corp., San Leandro, Calif.).

d. DETC Functional Assays

The DETC cell line, 7-17 was kindly provided by Dr. Wendy Havran (The Scripps Research Institute, La Jolla, Calif.). These cells were originally isolated by FACS of epidermal cell preparations from AKR mice (Kuziel et al, 1987). Cells were maintained in complete RPMI (with IL-2) and stimulated every 21 days with 5 μg/ml Con A. Only resting 7-17 DETCs (i.e., cells stimulated with Con A>7 days previously) were used in functional assays. 5×10⁵ cells were cultured in 96-well plates bound with 0.5 μg/ml anti-CD3 mAb (predetermined sub-optimal concentration) or 2 μg/ml anti-CD3 (predetermined optimal concentration) and 10 μg/ml anti-FLAG® M2 antibody. Approximately 30 minutes before plating cells, 650 ng of CD200.FLAG or BAP.FLAG was added to anti-CD3, anti-FLAG coated plates. Cells were cultured in complete RPMI (without IL-2) at 37° C. in 5% CO₂. After 72 hrs, cytokine levels were measured from culture supernatants using cytometric bead arrays (CBA; BD Biosciences, San Diego, Calif.) according to the manufacturer's protocol. To measure proliferation, cells were pulsed with 1 μCi/ml [³H]-thymidine at 72 hrs and assayed for thymidine uptake 16 hrs later.

e. Results

CD200R isoforms 1-4 were detected in freshly isolated ECs, with CD200R1 and CD200R2 having the highest levels of expression. Messenger RNA for CD200R1, R2, and R3 was detected in purified DETCs, with isoforms R1 and R2 preferentially expressed over the R3 isoform. CD200R1 expression was increased on purified LCs as compared to DETCs. CD200R1, R2, and R3 were detected in MCKII⁻/γδ TCR LCs, perhaps because of mast cell and/or basofphil contamination from the dermis or from mast cell precursors known to be present with in the epidermis (see, Kumamoto, et al. (2003) Blood 102:1654-1660).

EC suspensions prepared from CD200^{-/-} mice have significantly increased expression of CD1d, Cd11c, CD80, CD95 (FAS), CD178 (FASL), GR-1, F4/80, and OX-40L as compared to wild-type C57B6 mice. There was also significantly higher expression of MHC class II on LCs from CD200−/31 mice as compared to wild-type controls.

To determine if DETCs increased expression of CD200R upon activation. Epidermal cells from B6 mice were cultured on anti-CD3 mAb coated plates in the presence of IL-2. At various times, cells were harvested and γδ-TCR⁺ DETCs were stained for cell surface CD200R1 and analyzed by flow cytometry. A marked increase in CD200R1 expression was observed by 48 hrs post-activation, but maximum expression was observed at 72 hrs. To determine expression of the other CD200R isoforms, DETCs were purified by FACS to ≧99% purity, cultured on anti-CD3 coated plates in the presence of IL-2 and subjected to CD200R-specific qRT-PCR. Consistent with cell surface expression, DETCs markedly increased CD200R1 mRNA expression by 72 hrs after activation. In one of three experiments, activated DETCs also increased CD200R2 and CD200R3 mRNA expression; however, these increases were markedly diminished relative to increases in CD200R1 expression (FIG. 4). CD200R4 MRNA was not detected in either fresh or ex vivo activated DETCs.

Due to limitations in obtaining sufficient numbers of DETCs for functional studies, the DETC cell line 7-17 was used to assess the functional role of CD200-CD200R interactions. By several cellular and molecular criteria 7-17 cells have been shown to retain the properties of freshly isolated DETCs and are widely used in studies addressing DETC function (see, e.g., Havran et al (1991) Science 252:1430-1432; Matsue et al (1993) J. Immunol. 151:6012-6019; Matsue et al (1993) J. Invest. Dermatol. 101:543-548; Matsue et al (1993) J. Invest. Dermatol. 101:537-542; Edelbaum et al (1995) J. Invest. Dermatol. 105:837-843; Schuhmachers et al (1995) J. Invest. Dermatol. 105:225-230; Takashima et al (1995) J. Invest. Dermatol. 105:50S-53S; and Ono et al (1996) J. Dermatol. Sci. 11:89-96). To determine if 7-17 cells expressed CD200R1, resting cells (>7 days post Con A activation) were cultured on anti-CD3 coated plates in the presence of IL-2. At various times, cells were harvested for CD200R1 -specific staining. Similar to freshly isolated DETCs, 7-17 DETCs increased cell surface expression of CD200R1 upon activation. In contrast to freshly isolated DETCs, however, CD200R1 expression was observed as early as 24 hours after activation. By 72 hours, a population of presumably non-activated cells (decreased forward light scatter) remained negative for CD200R1 expression. To determine the expression pattern of all four CD200R isoforms, qRT-PCR was performed on both resting and anti-CD3 activated 7-17 cells. Consistent with cell surface expression, 7-17 cells showed an increase in CD200R1 mRNA upon activation. The level of CD200R1 mRNA increased an average of 8.8-fold by 72 hrs post-activation over 3 experimental replicates. mRNA for CD200R2 increased 1.8-fold by 72 hours; however, this increase was not statistically significant. In contrast to freshly isolated DETCs, resting 7-17 cells expressed low levels of CD200R4 mRNA, and upon activation, CD200R4 mRNA increased an average of 4.1-fold by 72 hours. Also, in contrast to freshly isolated DETCs, mRNA for CD200R2 was preferentially expressed in resting 7-17 cells.

To determine if CD200 signaling influences DETC function in vitro, 7-17 cells were activated with sub-optimal anti-CD3 mAb in the presence of immobilized CD200.FLAG fusion protein and measured both proliferation and cytokine secretion. 7-17 cells were cultured on microwell plates pre-coated with a sub-optimal amount of anti-CD3 antibody (predetermined concentration) and with CD200.FLAG fusion protein in the absence of IL-2. As a negative control, cells were cultured on plates pre-coated with both sub-optimal anti-CD3 and bacterial alkaline phosphatase (BAP).FLAG fusion protein. BAP.FLAG, approximately the same molecular weight as CD200.FLAG, was prepared in the same manner, and should not bind to DETCs. As a positive control, 7-17 cells were stimulated with an optimal amount of immobilized anti-CD3 antibody. Immobilized CD200 significantly inhibited the proliferative response of 7-17 cells to sub-optimal CD3 stimulation. Between 3- and 11-fold reductions in proliferation was observed when 7-17 DETCs were cultured on CD200-coated plates compared to BAP-coated plates over three replicate experiments. In addition, cytokine secretion was diminished when 7-17 cells were activated in the presence of CD200. A marked reduction in IL-2, TNFα, and IFNγ was observed in CD200-treated cells relative to BAP-treated cells in all three replicate experiments. The results for IL-5 and IL-10 were inconsistent, and IL-4, IL-6, IL-12 (p70), and MCP-1 were assayed but not consistently detected.

The above results support a finding that the CD200-CD200R interaction plays a regulatory role in both Langerhans and γδ+ T cell populations of the skin.

All citations herein are incorporated herein by reference to the same extent as if each individual publication, patent application, or patent was specifically and individually indicated to be incorporated by reference including all figures and drawings.

Many modifications and variations of this invention, as will be apparent to one of ordinary skill in the art, can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

1. A method of treating a condition or disorder associated with a hair follicle comprising administering to a subject an effective amount of an agonist of:
a) CD200; or
b) CD200R.

2. The method of claim 1, wherein the agonist is from the antigen binding site of an antibody that specifically binds to:
a) CD200; or
b) CD200R.

3. The method of claim 1, wherein the agonist comprises:
a) a polyclonal antibody;
b) a monoclonal antibody;
c) a humanized antibody, or a fragment thereof;
d) an Fab, F(ab′)₂, or Fv fragment;
e) a bispecific antibody;
f) a peptide mimetic of an antibody; or
g) a small molecule.

4. The method of claim 3, wherein the bispecific antibody specifically binds CD200R and an activating receptor.

5. The method of claim 1, wherein the agonist comprises:
a) a soluble polypeptide derived from an extracellular region of CD200, wherein the soluble polypeptide specifically binds to CD200R; or
b) a soluble polypeptide derived from an extracellular region of CD200R, wherein the soluble polypeptide specifically binds to CD200.

6. The method of claim 1, wherein the agonist comprises a nucleic acid.

7. The method of claim 6, wherein the nucleic acid encodes:
a) CD200 or CD200R; or
b) a soluble polypeptide derived from an extracellular region of CD200 or an extracellular region of CD200R.

8. The method of claim 1, wherein the condition or disorder comprises alopecia and the agonist ameliorates the alopecia or increases hair growth.

9. The method of claim 8, wherein the alopecia comprises:
a) scarring alopecia; or
b) non-scarring alopecia.
c) androgenetic alopecia (AGA);
d) alopecia areata (AA);
e) pseudopelade of Brocq (PB);
f) lichen planopilaris (LPP); or
g) fibrosing alopecia (FA).

10. The method of claim 1, wherein the condition or disorder comprises:
a) hair loss or baldness;
b) fibrosis in a dermal layer of the hair follicle;
c) intrafollicular edema;
d) apoptosis of a cell of the hair follicle;
e) infiltration of the hair follicle by an immune cell;
f) hair follicle depigmentation; or
g) excess hair.

11. The method of claim 1, wherein the agonist results in increased expression of:
a) insulin-like growth factor-1; or
b) interferon-gamma.

12. The method of claim 1, wherein the CD200 is expressed by:
a) an outer root sheath;
b) a keratinocyte;
c) a Langerhans cell;
d) a keratin-14 expressing cell; or
e) a hair follicle stem cell or transit amplifying cell.

13. A method of treating a disorder or condition associated with excess hair growth comprising administration of an antagonist of CD200 or CD200R.

14. A method of diagnosing a condition or disorder of a hair follicle comprising contacting a binding composition to a biological sample, wherein the binding composition specifically binds to:
a) CD200; or
b) CD200R,
and measuring or determining the specific binding of the binding composition to the biological sample.

15. The method of claim 14, wherein the biological sample is derived from a hair follicle of:
a) a tissue afflicted with a condition or disorder of the hair follicle; or
b) a control subject or non-afflicted tissue.

16. A kit comprising a compartment and:
a) the agonist of CD200 or CD200R; or
b) a nucleic acid that specifically hybridizes to a polynucleotide encoding CD200 or CD200R.
